SEQUENCE LISTING (14 sequences)

SEQ ID NO: 1 - protein, 5 aa, Artificial
  Dileucine Motif
  MISC_FEATURE (2)..(3): Xaa can be any naturally occurring amino acid
DXXLL

SEQ ID NO: 2 - protein, 6 aa, Artificial
  Dileucine Motif
  MISC_FEATURE (2)..(4): Xaa can be any naturally occurring amino acid
DXXXLL

SEQ ID NO: 3 - protein, 6 aa, Artificial
  Dileucine Motif
  MISC_FEATURE (2)..(4): Xaa can be any naturally occurring amino acid
EXXXLL

SEQ ID NO: 4 - protein, 6 aa, Artificial
  Dileucine Motif
  MISC_FEATURE (2)..(4): Xaa can be any naturally occurring amino acid
DXXXLI

SEQ ID NO: 5 - protein, 6 aa, Artificial
  Dileucine Motif
  MISC_FEATURE (2)..(4): Xaa can be any naturally occurring amino acid
EXXXLI

SEQ ID NO: 6 - protein, 5 aa, Artificial
  Dileucine Motif
  MISC_FEATURE (2)..(3): Xaa can be any naturally occurring amino acid
RXXLL

SEQ ID NO: 7 - protein, 5 aa, Artificial
  Dileucine Motif
  MISC_FEATURE (2)..(3): Xaa can be any naturally occurring amino acid
TXXLL

SEQ ID NO: 8 - protein, 7 aa, Mus musculus
SRRNRLL

SEQ ID NO: 9 - protein, 7 aa, Homo sapiens
SKRSRLL

SEQ ID NO: 10 - protein, 7 aa, Artificial
  GLUT4 Dileucine Motif
RRTPSLL

SEQ ID NO: 11 - protein, 7 aa, Artificial
  IRAP Dileucine Motif
PRGSRLL

SEQ ID NO: 12 - protein, 7 aa, Artificial
  VAMP4 Dileucine Motif
SERRNLL

SEQ ID NO: 13 - protein, 6 aa, Artificial
  Dileucine Motif
  MISC_FEATURE (2)..(4): Xaa can be any naturally occurring amino acid
RXXXLL

SEQ ID NO: 14 - protein, 6 aa, Artificial
  Dileucine Motif
  MISC_FEATURE (2)..(4): Xaa can be any naturally occurring amino acid
RXXXLI

(19) United States
(12) Patent Application Publication
(10) Pub. No.: US 2007/0066802 A1
(43) Pub. Date: Mar. 22, 2007

(54) Chimeric receptors with disrupted dileucine motifs

(76) Inventor: Terrance L. Geiger, Memphis, TN (US)

Correspondence Address: ST. JUDE CHILDREN'S RESEARCH HOSPITAL;OFFICE OF TECHNOLOGY LICENSING, 332 N. LAUDERDALE, MEMPHIS, TN, 38105, US

(21) Appl. No.: 10/568,235

(22) PCT Filed: Aug. 11, 2004
(86) PCT No.: PCT/US04/25930

§ 371 Date: Feb. 14, 2006

Related U.S. Application Data
(60) Provisional application No. 60/496,449, filed on Aug. 20, 2003.

(51) Int. Cl. C07K 14/74; C12P 21/06

(52) U.S. Cl. 530/350; 435/69.1; 435/320.1; 435/325

(57) ABSTRACT

A simple disruption of a dileucine motif in chimeric cell membrane receptors is taught. The disruption increases the capacity of the modified receptor to accumulate on the cell membrane relative to the unmodified receptor. The invention is particularly well suited for chimeric receptors comprising a portion of the CD28 protein that contains a dileucine motif.

### GOVERNMENT INTEREST

This invention was made in part with U.S. Government support under National Institutes of Health grant nos. AI49872 and CA21765. The U.S. Government may have certain rights in this invention.

### FIELD OF THE INVENTION

This invention relates to chimeric cell membrane receptors, particularly chimeric receptors derived in part from CD28.

### BACKGROUND

Cell membrane receptors are modular in nature, with various components and domains that can be mixed and matched to create chimeric receptors having a combination of desired attributes in a single protein. Components used to make chimeric receptors have been derived from a variety of receptors and other proteins including cytokine receptors, janus kinases, syk family tyrosine kinases, src family tyrosine kinases, growth factor receptors, antibodies, major histocompatibility complex (MHC), CD4, CD8, T cell receptor and homologues, antibody molecules, molecules involved in T cell transduction such as CD2, CD5, CD7, CD28, single-chain TCR and single-chain Fv.

Chimeric receptors have been designed and used for a variety of purposes. For example, chimeric co-stimulatory receptors have been designed for expression in cells to treat cancer, disease and viral infections. See U.S. Pat. No. 5,686,281. Many chimeric receptors utilize immune system components and have been designed to direct immune responses to particular desired targets such as tumors, viruses, or cells causing autoimmune disease. See, e.g. U.S. Pat. Nos. 6,451,314; 6,103,521; 6,083,751; 5,843,728; 5,837,544 and published U.S. Application Nos. 20030096778 and 20020137697.

The efficacy of chimeric receptors is dependent, at least in part, on the levels at which the receptors are present on the cell membrane. Any methods or techniques which could increase the level of these chimeric receptors on the membrane of the cell would increase the activity and efficacy of these receptors.

### SUMMARY OF THE INVENTION

The present invention provides a modification that can be made to any chimeric cell membrane receptor having a dileucine motif in its intracellular portion. The modification comprises a disruption of the dileucine motif which reduces or eliminates the capacity of the dileucine motif to induce internalization and loss of the chimeric protein from the cell membrane. By reducing internalization, this modification increases the steady state levels of the chimeric receptor present on the cell membrane.

In one aspect of the invention, a method for modifying a chimeric receptor to increase its capacity to accumulate in a cell membrane is provided. This method comprises disruption of the dileucine domain of the chimeric receptor. In another aspect, modified chimeric receptors having a disrupted dileucine motif are provided. These modified chimeric receptors include, but are not limited to, chimeric receptors having a CD4 or CD28 intracellular component that includes a disrupted dileucine motif.

In yet another aspect of the invention, a CD28 protein and portions thereof having a disrupted dileucine motif are provided as a template for generating a modified chimeric receptor.

In yet another aspect of the invention, cells having modified chimeric receptors on their membrane are provided. In particular, T-cells having modified chimeric receptors are provided.

The dileucine motif which is the target of modification according to the present invention is a motif within transmembrane proteins containing two leucines that recognizes components on the cytosolic face of membranes that promote sorting to endosomes and lysosomes. This motif is characterized structurally by the presence of two leucines in succession, or less commonly by a leucine followed by an isoleucine. This motif can be altered in any desired way, including removing amino acids, adding amino acids or substituting amino acids, to disrupt its internalization function. A preferred alteration to disrupt the internalization is a conservative substitution of one or both of the leucines (or leucine-isoleucine combination) within this motif.

### DESCRIPTION OF THE SEQUENCE LISTING

SEQ ID No. 1 is the general dileucine motif AspXaaXaaLeuLeu, where Xaa can be any naturally occurring amino acid.
SEQ ID No. 2 is the general dileucine motif AspXaaXaaXaaLeuLeu, where Xaa can be any naturally occurring amino acid.
SEQ ID No. 3 is the general dileucine motif Glu XaaXaaXaaLeuLeu, where Xaa can be any naturally occurring amino acid.
SEQ ID No. 4 is the general dileucine motif AspXaaXaaXaaLeuIle, where Xaa can be any naturally occurring amino acid.
SEQ ID No. 5 is the general dileucine motif Glu XaaXaaXaaLeulle, where Xaa can be any naturally occurring amino acid.
SEQ ID No. 6 is the general dileucine motif ArgXaaXaaLeuLeu, where Xaa can be any naturally occurring amino acid.
SEQ ID No. 7 is the general dileucine motif ThrXaaXaaLeuLeu, where Xaa can be any naturally occurring amino acid.
SEQ ID No. 8 is the murine CD28 dileucine motif SerArgArgAsnArgLeuLeu
SEQ ID No. 9 is the human CD28 dileucine motif SerLysArgSerArgLeuLeu
SEQ ID No. 10 is the GLUT4 dileucine motif ArgArgThrProSerLeuLeu
SEQ ID No. 11 is the IRAP dileucine motif ProArgGlySerArgLeuLeu
SEQ ID No. 12 is the VAMP4 dileucine motif SerGluArgArgAsnLeuLeu
SEQ ID No. 13 is the general dileucine motif ArgXaaXaaXaaLeuLeu, where Xaa can be any naturally occurring amino acid.
SEQ ID No. 14 is the general dileucine motif ArgXaaXaaXaaLeuIle, where Xaa can be any naturally occurring amino acid.

### DESCRIPTION OF THE FIGURES

FIG. 1: Chimeric receptor structure and sequence of the dileucine motif. Chimeric constructs were created by linking components in a cassette fashion. Extracellular and transmembrane domains are derived from the MHC class I H-2K^{b} molecule. The murine CD28 and TCR-ζ cytoplasmic tails were attached as described in Geiger, T. L. et al., “Integrated src kinase and costimulatory activity enhances signal transduction through single-chain chimeric receptors in T lymphocytes”, Blood 98: 2364-2371 (2001). PCR mutagenesis was used to introduce the leucine to glycine change in the CD28 tail. This corresponds to an L184G and L185G conversion in the CD28 sequence (Genbank accession NP_{—}031668).

### DETAILED DESCRIPTION OF THE INVENTION

### DEFINITIONS

CD4: A protein expressed on the surface of T-lymphocytes and some other cell types that supports signal transduction, presumably by binding the src kinase lck. Genbank accession BC039137 (mouse); NM_{—}000616 (homo sapiens); see also Tourvieille, B. et al., “Isolation and sequence of L3T4 complementary DNA clones: expression in T cells and brain”, Science 234 (4776): 610-614 (1986); Maddon, P. J. et al., “The isolation and nucleotide sequence of a cDNA encoding the T cell surface protein T4: a new member of the immunoglobulin gene family”, Cell 42 (1): 93-104 (1985).

CD28: A protein that provides supplementary signals to T-cells when stimulated by its receptor, enhancing activation and promoting cell survival and proliferation. Genbank accession NM_{—}007642 (mouse); NM_{—}006139 (homo sapiens); see also Gross, J. A. et al., “The murine homologue of the T lymphocyte antigen CD28. Molecular cloning and cell surface expression”, J. Immunol. 144 (8): 3201-3210 (1990); Aruffo, A. and Seed, B, “Molecular cloning of a CD28 cDNA by a high-efficiency COS cell expression system”, Proc. Natl. Acad. Sci. U.S.A. 84 (23): 8573-8577 (1987).

Dileucine motif: A motif within the cytoplasmic portion of transmembrane proteins containing two leucines that recognizes components on the cytosolic face of membranes that promote sorting to endosomes and lysosomes. This motif is characterized structurally by the presence of two leucines in succession, or less commonly by a leucine followed by an isoleucine. Most dileucine motifs fall within one of the following general formulae AspXaaXaaLeuLeu (SEQ ID No. 1), AspXaaXaaXaaLeuLeu (SEQ ID No. 2), Glu XaaXaaXaaLeuLeu (SEQ ID No. 3), ArgXaaXaaXaaLeuLeu (SEQ ID No. 13), AspXaaXaaXaaLeuIle (SEQ ID No. 4), Glu XaaXaaXaaLeuIle (SEQ ID No. 5), or ArgXaaXaaXaaLeuIle (SEQ ID No. 14) where Xaa can be any naturally occurring amino acid. Occasionally other amino acids can substitute for the Asp in the AspXaaXaaLeuLeu (SEQ ID No. 1) motif, such as Arg (SEQ ID. No. 6) or Thr (SEQ ID No. 7). This is the case for CD28 whose dileucine motif is SerArgArgAsnArgLeuLeu (SEQ ID No. 8) in mouse and SerLysArgSerArgLeuLeu (SEQ ID No. 9) in humans. Another example is GLUT4, which has a ArgArgThrProSerLeuLeu (SEQ ID No. 10) dileucine motif. Yet another example is RAP, which has a ProArgGlySerArgLeuLeu (SEQ ID No. 11) dileucine motif. Yet another example is VAMP4, which has a SerGluArgArgAsnLeuLeu (SEQ ID No. 12) dileucine motif.

Disrupt, or disrupting: With respect to the dileucine motif, the terms “disrupt”, “disruption” or “disrupting” mean altering the dileucine motif in such a way that it no longer promotes the cellular internalization of the protein it is a part of Such disruptions include, but are not necessarily limited to, adding one or more amino acids to the dileucine motif, removing one or more amino acids from the dileucine motif, substituting amino acids within the dileucine motif, or some combination thereof. Effective disruption of a dileucine motif can be assayed functionally using conventional techniques by determining the impact of the disruption on expression levels of the chimeric protein or by assessing the effect of the disruption on rates of internalization of the chimeric protein, either spontaneous internalization or internalization after stimulation or crosslinking of the chimeric protein.

### DETAILED DESCRIPTION

The present invention is based in part on the identification of a dileucine motif in CD28 and a recognition of its role in internalization of chimeric CD28 receptors which incorporate this motif. By disrupting this motif in a chimeric CD28 receptor, the inventors were able to increase the levels of this receptor present on the membrane of expressing cells (see Example 1).

This finding is contemplated to be generally applicable to any chimeric receptor having a dileucine motif in its intracellular portion. By disrupting this motif such that it no longer promotes cellular internalization, the resulting modified chimeric receptor can accumulate on the cell membrane at higher levels than its unmodified counterpart Thus the present invention provides a way of effectively increasing the activity of a chimeric receptor by increasing its steady state levels on the cell membrane.

Disruptions which can be made to the dileucine motif include, but are not necessarily limited to, adding one or more amino acids to the dileucine motif, removing one or more amino acids from the dileucine motif, substituting amino acids within the dileucine motif, and any combination of these. A preferred disruption is the substitution of one or both of the leucines (or the leucine/isoleucine combination for motifs corresponding to SEQ ID Nos. 4 and 5)) in the dileucine motif with an amino acid of similar size and charge (conservative substitution) such as glycine or alanine.

Chimeric receptors which may benefit from application of this invention can be identified by the presence of a dileucine motif having a sequence corresponding to one of SEQ ID Nos. 1-12, or minor variants thereof, in their intracellular portion. Once identified, the dileucine motif may be disrupted using any desired means to generate a modified chimeric receptor that will accumulate on the cell membrane at higher levels than the unmodified receptor.

Modified chimeric receptors of the invention represent an improvement over their unmodified counterparts and can be used for the same purpose. For example, chimeric receptors have been designed for the treatment of cancer, infectious disease, autoimmune disease and other immune disorders. See U.S. Pat. Nos. 6,451,314; 6,103,521; 6,083,751; 5,843,728; 5,837,544 and published U.S. Application Nos. 20030096778 and 20020137697.

Many chimeric receptors utilize the intracellular region of CD28 which contains the dileucine motif. See Example 1 and U.S. Pat. Nos 6,103,521 and 6,083,751. To facilitate the generation of modified versions of these receptors in accordance with the invention, a modified form of the CD28 protein having a disrupted dileucine motif may also be made according to the invention. A modified form of human CD28 can be made according to the present invention by disrupting the dileucine motif SerLysArgSerArgLeuLeu (SEQ ID No. 9). A modified form of murine CD28 can be made according to the present invention by disrupting the dileucine motif SerArgArgAsnArgLeuLeu (SEQ ID No.8). A whole modified CD28 protein or appropriate portions thereof comprising the disrupted dileucine motif may be used as the source of an intracellular portion of these chimeric receptors.

A cell containing at least one modified chimeric receptor on its membrane is also contemplated as part of the present invention. Any desired type of cell capable of tolerating a modified chimeric receptor in its membrane could be used in this aspect of the invention. Such a cell can be engineered to express a modified chimeric receptor of the invention using conventional methodology. Of particular use in this aspect is a T-cell having on its membrane a modified chimeric receptor that allows the T-cell to target specific antigens, including antigens not normally recognized by the immune system (see Example 1).

The present invention may be better understood by reference to the following non-limiting example. This example is presented in order to more fully illustrate the invention through the description of a particular embodiment. This example should in no way be construed as limiting the scope of the invention.

### EXAMPLES

### Example 1

### Identification of a CD28 Dileucine Motif that Suppresses Singe-Chain Chimeric T-Cell Receptor Expression and Function

Introduction

T cells transgenically modified to express genetically engineered chimeric receptors (receptor-modified T cells, RMTC) can target antigens not normally recognized by the immune system (Geiger, T. L., and Jyothi, M. D., “Development and application of receptor-modified T lymphocytes for adoptive immunotherapy”, Transfus Med Rev. 15: 21-34 (2001); Sadelain, M. et al., “Targeting tumours with genetically enhanced T lymphocytes”, Nat Rev Cancer 3:35-45 (2003); Abken, H. et al., “Chimeric T-cell receptors: highly specific tools to target cytotoxic T-lymphocytes to tumour cells”, Cancer Treat Rev. 23:97-112 (1997)). The chimeric receptors that redirect these RMTC against their targets functionally substitute for the T cell receptor (TCR). They recognize target antigen through an extracellular antigen-recognition domain, such as a single-chain Fv fragment, and signal the RMTC through a linked TCR-derived signal transduction domain, typically from the TCR-ζ chain. RMTC have shown therapeutic potency in model systems, selectively targeting cancerous, infected, and autoreactive T cells, and have not shown significant toxicity in phase I clinical trials (Brentjens, R. J. et al., “Eradication of systemic B-cell tumors by genetically targeted human T lymphocytes co-stimulated by CD80 and interleukin-15”, Nat Med. 9:279-286 (2003); Haynes, N. M. et al., “Rejection of syngeneic colon carcinoma by CTLs expressing single-chain antibody receptors codelivering CD28 costimulation”, J Immunol. 169:5780-5786 (2002); Kershaw, M. H. et al., “Dual-specific T cells combine proliferation and antitumor activity”, Nat Biotechnol. 20:1221-1227 (2002); Mitsuyasu, R. T. et al., “Prolonged survival and tissue trafficking following adoptive transfer of CD4zeta gene-modified autologous CD4(+) and CD8(+) T cells in human immunodeficiency virus-infected subjects”, Blood 96:785-793 (2000)).

Although engineered surrogate receptors can redirect therapeutic T cells, their effectiveness in doing this may be limited by the limited signal they can transduce. Co-receptor and costimulatory signals, normally provided to T cells when they interact with a “professional” antigen-presenting cell, will often not be available to RMTC engaging a ligand on a tumor or other target cell with a chimeric receptor (Lenschow, D. J. et al., “CD28/B7 system of T cell costimulation”, Annu Rev Immunol. 14:233-258 (1996); Watts, T. H. and DeBenedette, M. A., “T cell co-stimulatory molecules other than CD28” Curr Opin Immuno. 11:286-293 (1999)). These signals can promote T cell survival, proliferation, and effector function, and may therefore be critical for RMTC function. To overcome this limitation, we and others have developed single-chain chimeric receptors that incorporate modular signal transduction subunits derived from the TCR, costimulatory, and/or co-receptor molecules (Haynes, N. M. et al., “Rejection of syngeneic colon carcinoma by CTLs expressing single-chain antibody receptors codelivering CD28 costimulation”, J Immunol. 169:5780-5786 (2002); Geiger, T. L. et al., “Integrated src kinase and costimulatory activity enhances signal transduction through single-chain chimeric receptors in T lymphocytes”, Blood 98: 2364-2371 (2001); Finney, H. M. et al., “Chimeric receptors providing both primary and costimulatory signaling in T cells from a single gene product”, J Immunol. 161:2791-2797 (1998); Hombach, A. et al., “Tumor-specific T cell activation by recombinant immunoreceptors: CD3 zeta signaling and CD28 costimulation are simultaneously required for efficient IL-2 secretion and can be integrated into one combined CD28/CD3 zeta signaling receptor molecule”, J Immunol. 167:6123-6131 (2001); Maher, J. et al., “Human T-lymphocyte cytotoxicity and proliferation directed by a single chimeric TCRzeta/CD28 receptor” Nat Biotechnol. 20:70-75 (2002)). The receptor structure most commonly analyzed includes the signaling domain of the CD28 costimulatory molecule genetically linked to the cytoplasmic tail of the TCR ζ chain. In several studies, RMTC that expressed chimeric receptors including a CD28-ζ signaling region, when compared with those including only ζ, showed improved functional responses in vitro and in vivo.

We have been interested in using RMTC to specifically target T lymphocytes that are pathologic in transplant or other settings. The defining feature of a pathologic T lymphocyte is the specificity of its TCR. In transplantation, these TCRs are generally directed against allogeneic MHC or syngeneic MHC coupled with minor histocompatability antigens (Warren, E. H. et al., “Minor histocompatibility antigens as targets for T-cell therapy after bone marrow transplantation. Curr Opin Hematol 5:429-433 (1998); Waldmann, H. et al., “What can be done to prevent graft versus host disease?” Curr Opin Immunol. 6:777-783 (1994)). To specifically redirect RMTC against pathologic class I MHC-restricted T lymphocytes in murine models of transplantation, we developed chimeric receptors that include the class I MHC K^{b} molecule extracellular and transmembrane domains linked to either a murine ζ or CD28-ζ signaling tail (Geiger, T. L. et al., “Integrated src kinase and costimulatory activity enhances signal transduction through single-chain chimeric receptors in T lymphocytes”, Blood 98: 2364-2371 (2001)). The K^{b} extracellular region serves as bait for K^{b}-restricted pathologic T cells, whereas the signaling domain activates the RMTC, inducing effector functions. Biochemical analysis of receptor-mediated signal transduction in K^{b}-CD28-ζ transduced T cell hybridomas, when compared with K^{b}-ζ transduced cells, demonstrated enhanced receptor phosphorylation and calcium flux. Further, the added CD28 domain allowed direct receptor association with the src kinase p561ck, which is critically involved in initiating and sustaining receptor-mediated signal transduction. T-cell hybridomas expressing the K^{b}-CD28-ζ receptor also showed increased IL-2 production and signaling sensitivity.

In contrast to the enhanced function of the K^{b}-CD28-ζ receptor in immortaized T cell hybridomas, when we transduced primary T lymphocytes with this or the K^{b}-ζ receptor, we did not observe significant differences in chimeric receptor mediated functional responses (Nguyen, P. and Geiger T. L. “Antigen-specific targeting of CD8(+) T cells with receptor-modified T lymphocytes” Gene Ther. 10:594-604 (2003)). We further observed a 2-4 fold decreased expression level of the K^{b}-CD28-ζ receptor in primary T cells when compared with the K^{b}-ζ receptor. Interestingly, decreased expression of CD28-ζ containing receptors when compared with otherwise identical ζ-containing receptors is reflected in results reported by others, although this decrease has not previously been quantitatively analyzed (Finney, H. M. et al., “Chimeric receptors providing both primary and costimulatory signaling in T cells from a single gene product”, J Immunol. 161:2791-2797 (1998); Hombach, A. et al., “Tumor-specific T cell activation by recombinant immunoreceptors: CD3 zeta signaling and CD28 costimulation are simultaneously required for efficient IL-2 secretion and can be integrated into one combined CD28/CD3 zeta signaling receptor molecule”, J Immunol. 167:6123-6131 (2001); Maher, J. et al., “Human T-lymphocyte cytotoxicity and proliferation directed by a single chimeric TCRzeta /CD28 receptor” Nat Biotechnol. 20:70-75 (2002)).

The inclusion of the CD28 costimulatory region in chimeric receptor signal transduction units therefore has conflicting effects. It provides an enhanced signal into RMTC, but simultaneously diminishes receptor surface expression and thereby limits the magnitude and/or duration of this signal. This decreased expression may be particularly significant with the K^{b}-CD28-ζ receptor, which engages its cognate TCR ligand in a low affinity interaction (Alam, S. M. et al. “Qualitative and quantitative differences in T cell receptor binding of agonist and antagonist ligands” Immunity. 10:227-237 (1999)).

To determine potential sources for the reduced expression of the K^{b}-CD28-ζ receptor we analyzed the sequence of the murine CD28 cytoplasmic tail. We noticed there a non-canonical dileucine internalization motif (Bonifacino, J. S. and Traub, L. M. “Signals for Sorting of Transmembrane Proteins to Endosomes and Lysosomes”. Annu Rev Biochem. 72:395-447 (2003)). This motif had not been previously studied in regards to CD28 function, though dileucine motifs have been well characterized in other proteins. Dileucine motifs bind AP or GGA adaptor proteins, and thereby promote receptor internalization. To clarify the role of the CD28 dileucine motif in CD28-ζ chimeric receptor function we inactivated it in the K^{b}-CD28-ζ receptor. We found that mutating the essential leucines in the motif to glycines increases surface expression of the K^{b}-CD28-ζ receptor 2-5 fold when compared with the unmutated receptor. Further K^{b}-CD28[L→G]-ζ modified T cells showed increased sensitivity in proliferation, cytokine production, and cytolysis studies when compared with K^{b}-CD28-ζ modified T cells, and were highly effective in eliminating antigen-specific target T lymphocytes in vivo. This study therefore identifies a previously undescribed dileucine motif within the murine CD28 tail, demonstrates a specific role for this dileucine motif in limiting chimeric receptor function in RMTC, and illustrates how protein engineering may be used to modify specific motifs within multi-domain chimeric receptors to optimize their expression and function. We additionally provide the first evidence that RMTC may be used to target antigen-specific CD8⁺ T cells in-vivo, suggesting a potential new use for RMTC in the generation of transplant tolerance.

Materials and Methods

Constructs: Synthesis and sequences of the chimeric constructs are as described in Geiger, T. L. et al., “Integrated src kinase and costimulatory activity enhances signal transduction through single-chain chimeric receptors in T lymphocytes”, Blood 98: 2364-2371 (2001). Briefly, cDNA fragments encoding the extracellular and transmembrane domain of the H-2K^{b} molecule, and the cytoplasmic tails of murine CD28 and ζ were isolated by PCR from cDNA clones or splenic cDNA. The dileucine mutation was introduced by PCR mutagenesis. Flanking restriction sites were added to the fragments by PCR, and the fragments linked. Assembled constructs were subcloned into the MSCV-I-GFP retroviral vector (gift of Dr. Elio Vanin; Persons D. A. et al., “Retroviral-mediated transfer of the green fluorescent protein gene into murine hematopoietic cells facilitates scoring and selection of transduced progenitors in vitro and identification of genetically modified cells in vivo”, Blood. 90:1777-1786 (1997)). Fidelity of construct DNA sequences was confirmed by sequencing at the St. Jude Hartwell Center for Biotechnology.

Mice, cells, and antibodies: TG-B mice (Geiger, T. et al. “T-Cell Responsiveness to an Oncogenic Peripheral Protein and Spontaneous Autoimmunity in Transgenic Mice” Proc Natl Acad Sci USA. 89:2985-2989 (1992)), transgenic for a rearranged SV40-T/H-2K^{k} restricted TCR, were bred >20 generations with B10.BR mice and used as a source of CD8⁺ T cells for transducing constructs. OT-1 mice (Jackson Laboratories, Bar Harbor, Me.), transgenic for a rearranged ovalbumin 257-264/H-2K^{b} restricted TCR, were used as a source of target cells. C57BL/6J-Prkdc^{scid}/SzJ mice (Jackson Laboratories) were used as adoptive transfer recipients. Antibodies used include: clone B20.1 anti-mouse V_{α}2 (Pharmingen, San Diego, Calif.); clone 2C11 anti-mouse CD3ε; clone AF6-88.5 anti-mouse H-2K^{b} (Pharmingen and gift of M. Blackmann); goat anti-mouse IgG (Jackson Laboratories); goat anti-rat IgG (Jackson Laboratories).

Retroviral transduction and T cell culture: Retrovirus was produced as described (Ausubel, F. M. et al., “Current Protocols in Molecular Biology”. New York: John Wiley and Sons; 1989). Briefly, 10 μg of chimeric receptor constructs and 10 μg of the retrovirus helper DNA construct PEQPAM (gift of Dr. John Cleveland) were cotransfected into 293-T cells by calcium phosphate precipitation. At 16 hours the cells were washed and cultured in Dulbecco Modified Eagle medium (DMEM)/10% fetal calf serum (FCS) for 48 hours. Supernatant was collected twice daily and used to infect GP+E86 retroviral producer cells in the presence of 8 μg/mL polybrene. Transduced GP+E86 cells were flow cytometrically sorted for the presence of green fluorescence protein (GFP) and expanded. To transduce T lymphocytes, isolated lymph node cells were stimulated with soluble CD3 and CD28 specific antibodies in the presence of 2 ng/mL rmIL-2 (R&D Systems, Minneapolis, Minn.) for 2 days. Medium was removed, replaced with cleared supernatant from the GP+E86 retroviral producer cells and 8 μg/mL polybrene, and the cells were spun at 1800 rpm for 90 minutes in a Jouan CR422 tabletop centrifuge. On day 4, transduced T cells were sorted for expression of GFP and CD8 and expanded by culturing in EHAA medium (Biosource International, Camarillo, Calif.) in the presence of rmIL-2 for up to 5 days. The cells were re-stimulated every 7-10 days using 2 μg/mL concanavalinA (conA; Sigma, St Louis, Mo.), 2×10⁶/mL 3000 rad irradiated syngeneic splenocytes, and 2 ng/mL rmIL-2. Transduced cells were washed and assayed 5-6 days after stimulation. Assays were performed in the absence of exogenously added IL-2.

Proliferation: The designated concentration of purified AF6-88.5 antibody was loaded onto goat anti-mouse IgG coated wells in 96 well plates. 5×10⁴ transduced T cells and 2.5×10⁵ 2,500-rad irradiated syngeneic B10.BR splenocytes were added per well. After 2 days, the cells were pulsed with 1 μCi ³H-thymidine for 16 hours and harvested onto filtermats. Proliferation was measured by liquid scintillation counting of incorporated ³H. All samples were analyzed in triplicate and means plotted.

Cytotoxicity assay: Receptor-modified T cells, day 5-6 post-stimulation, were incubated overnight in medium to which was added 50 μg/mL or the designated concentration of ovalbumin 257-264 peptide (St. Jude Hartwell Center for Biotechnology) in PBS or control phosphate buffered saline (PBS) diluent, washed 3 times, and resuspended in medium. Effector cells were incubated with approximately 10⁵ OT-1 target T cells at the designated ratio. Primary T cell targets were isolated from OT-1 TCR transgenic lymph node cells or splenocytes by panning on goat anti-mouse IgG coated plates or by nylon wool. After 5-6 hours coincubation, 5,000-10,000 6 μm fluorescent TruCount beads (Becton Dickinson, Franklin Lakes, N.J.) were added. Samples were stained for V_{α}2, washed once, and analyzed by flow cytometry as described (Nguyen, P. and Geiger T. L. “Antigen-specific targeting of CD8(+) T cells with receptor-modified T lymphocytes” Gene Ther. 10:594-604 (2003)). Viable target T cells could be distinguished from effector T cells by the presence of V_{α}² and the absence of GFP. Absolute target cell numbers were determined by normalization of cellular events with the TruCount bead events. Percent specific cytotoxicity was determined as 100×(1-viable target cell count after incubation with peptide pulsed effectors/viable target cell count after incubation with unpulsed effectors). In all experiments parallel cultures of target cells in the absence of effector cells were simultaneously performed. Essentially identical results were obtained when cytotoxicity was alternatively calculated as 100×(1-viable target cell count after incubation with peptide pulsed effectors/viable target cell count after incubation without effectors). All samples were analyzed in pentuplicate.

Cytokine analysis: IFN-γ was analyzed using a Bioplex assay (Bio-Rad, Hercules, Calif.). A 96 well filter plate was prewet and approximately 3000 analytical beads added per well. Dilutions of standards or experimental samples were added to the beads and incubated for 1 h RT. Supernatant was aspirated, beads washed, and then incubated for 1 h with biotinylated anti-IFN-γ detection antibody. After washing detection was performed by staining with streptavidin-phycoerythrin (PE) and fluorescence analysis with a Bioplex plate reader (Bio-Rad).

In-vivo cytotoxicity: Peripheral lymph nodes from OT-1 mice were harvested, labeled with 5 μM carboxyfluorescein succinimidyl ester (CFSE, Molecular Probes, Eugene, Oreg.), and washed 3× with Hank's buffered sodium saline (HBSS). Approximately 10⁷ cells were injected intravenously into the retro-orbital plexus of a recipient mouse. Shortly after, approximately 10⁷ RMTC were injected into the alternate retro-orbital plexus. 24 h after injection, spleen and lymph nodes were harvested, and a single cell suspension prepared, stained with PE labeled anti-V_{α}2, and analyzed by flow cytometry.

Statistics: Standard deviations and paired 2-sided t-tests were calculated using Excel spreadsheet software. Error bars correspond to ±1 standard deviation.

Results

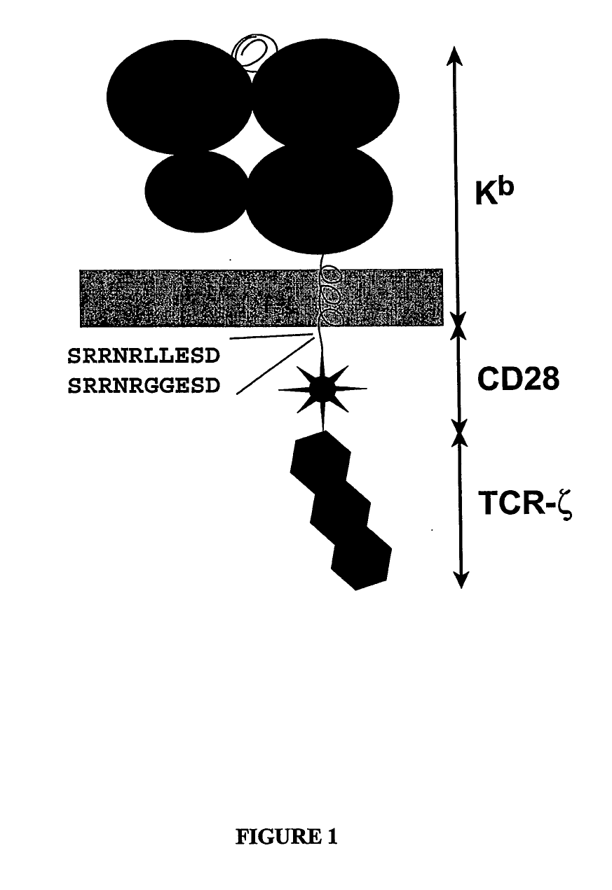

Design and expression of chimeric receptors. The wild-type K^{b}-CD28- [symbol] and dileucine-mutated K^{b}-CD28[L→G]-□ receptors included the H-2K^{b} extracellular and transmembrane domains, linked to the cytoplasmic domains of □ or CD28-□ [symbol] (FIG. 1). Constructs were subcloned into the MSCV-I-GFP retroviral vector, which includes an IRES linked green fluorescent protein (GFP) gene. Retrovirus-rich supernatant was produced and used to transduce primary CD8⁺ T lymphocytes. Transduction efficiencies of 15-50% were typically observed.

In order to determine the role of the dileucine motif in chimeric receptor expression, we flow cytometrically sorted CD8⁺GFP⁺ cells transduced with either the K^{b}-CD28-ζ or K^{b}-CD28[L→G]-ζ receptor and stained them with a K^{b}-specific antibody. Cells bearing the receptor mutated for the dileucine motif showed a 2-5 fold increase in receptor level compared with wild type receptor in several independent transduction experiments. This increased receptor expression did not result from increased transcription of the mutated when compared with the wild-type receptor. When chimeric receptor expression level was analyzed as a function of the level of linked and cotranscribed GFP, this 2-5 fold increase in expression level was consistently seen regardless of the amount of GFP present in individual cells. These results therefore demonstrate that the CD28 dileucine motif in chimeric receptors significantly restricts the level of surface chimeric receptor, while its disruption enhances surface expression.

Functional response of RMTC. A substantial body of data has demonstrated that the T cell response to stimulation will vary with the intensity and duration of the stimulus received (Iezzi, G. et al., “The duration of antigenic stimulation determines the fate of naive and effector T cells”, Immunity. 8:89-96 (1998); Lanzavecchia, A. et al., “From TCR engagement to T cell activation: a kinetic view of T cell behavior”, Cell, 96:14 (1999)). This implies that the increased expression of dileucine mutated chimeric receptors should result in improved signaling compared with unmutated receptors. However the role of the dileucine motif in CD28 signaling has not been established, and it was possible that disruption of this motif would cripple signal transduction. In order to determine the functional impact of the dileucine mutation on T cell functional responses we first measured T cell proliferation after stimulation through the K^{b}-CD28- [symbol] or K^{b}-CD28[L→G]-□ receptors. K^{b}-CD28- [symbol] , K^{b}-CD28[L→G]- [symbol] and retroviral vector modified T cells responded equivalently to a control, non-specific mitogen, concanavalin A, demonstrating that receptor expression did not adversely impact the ability of the transduced cells to proliferate. In contrast, T cells transduced with the dileucine mutated receptor proliferated significantly better than wild-type receptor-transduced T cells in response to chimeric receptor-specific stimulation. Therefore the CD28 dileucine motif functionally restricts chimeric receptor activity, and this restriction is alleviated by the L→G mutation.

To determine whether the enhanced function of RMTC expressing the mutated receptor extended to the production of effector cytokines, we analyzed IFN-γ release by RMTC. Stimulation with chimeric receptor-specific antibody induced >3.5 fold more IFN-γ in K^{b}-CD28[L→G]-ζ RMTC than in K^{b}-CD28-ζ RMTC. Thus disabling the dileucine motifs improves RMTC effector cytokine response.

The effector function of RMTC most often required for immunotherapy is target cell cytolysis. We have previously demonstrated that K^{b}-CD28-ζ modified RMTC antigen-specifically kill K^{b}-restricted target T cells with a similar efficiency as K^{b}-ζ modified cells. To compare the cytolysis efficiency of K^{b}-CD₂₈-ζ and K^{b}-CD28[L→G]-ζ RMTC we analyzed their ability to lyse transgenic OT-1 T cells. The OT-1 TCR recognizes the 257-264 peptide of ovalbumin (SIINFEKL) complexed with the chimeric receptor's extracellular K^{b} domain (Nguyen, P. and Geiger T. L. “Antigen-specific targeting of CD8(+) T cells with receptor-modified T lymphocytes” Gene Ther. 10:594-604 (2003)). We pulsed RMTC with this peptide or diluent, washed them, and coincubated the peptide or control pulsed RMTC with purified OT-1 TCR tansgenic T lymphocytes to analyze specific cytolysis.

We first examined the relationship of RMTC dose to cytolytic response. Equivalent specific lysis of OT-1 T cells occurred with approximately 3-9 fold fewer peptide-pulsed K^{b}-CD28[L→G]-ζ RMTC compared with K^{b}-CD28-ζ RMTC. This demonstrates an increased efficiency of lysis by RMTC transduced with the dileucine-mutated chimeric receptor. To better define how limitations in the quantity of chimeric receptor-ligand present on individual RMTC influences lytic potency, we varied the concentration of antigenic peptide used to peptide-pulse the RMTC. Fewer chimeric receptors would be expected to incorporate the ovalbumin peptide when lower antigen concentrations are used for pulsing. Our analysis showed that the K^{b}-CD28[L→G]-ζ RMTC lysed OT-1 target cells equivalently to the K^{b}-CD28-ζ RMTC when pulsed with 5-20 fold lower concentrations of antigenic peptide. This further demonstrates that the chimeric receptor dileucine mutation significantly increases the lytic potency of effector RMTC.

In-vivo targeting of antigen-specific T cells by RMTC. Peptide-pulsed K^{b}-CD28[L→G]-ζ or K^{b}-CD28-ζ RMTC efficiently and antigen-specifically lysed target OT-1 T cells in-vitro. To determine the feasibility of similarly targeting these cells in vivo, we labeled lymph node cells from OT-1 TCR transgenic mice with the fluorescent marker CFSE, and adoptively transferred them into SCID mice prior to the transfer of peptide-pulsed or unpulsed K^{b}-CD28[L→G]-ζ or K^{b}-CD28-ζ RMTC. 24 hours later we determined by flow cytometry the number of residual OT-1 cells present in the spleens and lymph nodes (LN) of treated mice. The CFSE⁺ OT-1 target cells were readily distinguished from the GFP⁺ RMTC by their fluorescence intensity and scatter characteristics. To distinguish ovalbumin-specific T cells from B-, non-specific T-, and other cell types present amongst the CFSE⁺ transferred cells, we stained the post-treatment splenocytes or lymph node cells with an anti-V_{α}2 antibody that recognizes ovalbumin-specific OT-1 T cells. The V_{α}2⁻-CFSE⁺ cell population, which is not specific for ovalbumin and should not be targeted by the RTMC, was, as expected, not significantly affected by peptide pulsed compared with unpulsed K^{b}-CD28[L→G]-ζ or K^{b}-CD28-Λ RMTC. In contrast, a significant loss of V_{α}2⁺CFSE⁺ OT-1 target T cells in both spleen and LN was seen in mice treated with peptide-pulsed compared with unpulsed RMTC. To control for the efficiency of adoptive transfer, we normalized the number of V_{α}2⁺CFSE⁺ cells detected to the number of unaffected V_{α}2⁻CFSE⁺ cells. With this normalization we could calculate that the peptide pulsed K^{b}-CD28-ζ and K^{b}-CD28[L→G]-ζ RMTC depleted 93±4% and 98±0.4% of V_{α}2⁺ OT-1 cells from the LN, and 96±3% and 99±0.1% from the spleen respectively. The K^{b}-CD28[L→G]-ζ RMTC consistently performed better than the K^{b}-CD28-ζ RMTC in 3 of 3 independent experiments in which splenocytes were analyzed and in 2 of 2 independent experiments analyzing lymph node cells. Cumulative data from these experiments, however, only showed a statistically significant difference for splenocytes (p=0.01, n=9 per treatment group for splenocytes; p=0.27, n=6 per treatment group for LN cells). These results therefore demonstrate that both K^{b}-CD28[L→G]-ζ and K^{b}-CD28-ζ RMTC are effective in depleting CD8⁺ antigen-specific T cells in vivo, show that RMTC expressing the dileucine mutated receptor are more active in vivo, and suggest that RMTC may be effective in inducing transplantation tolerance by eliminating pathologic alloreactive T cells.

Discussion

Immunotherapeutically targeting T lymphocytes against pathologic cell types is a primary objective of cellular immunotherapy. Adoptively transferred therapeutic T lymphocytes are long-lived, can migrate throughout the body, and express a variety of therapeutically useful effector functions. Further, clinically administered antigen-specific T cells have shown promise in the treatment of infectious diseases and cancer (Heslop, H. E. and Rooney, C. M. “Adoptive cellular immunotherapy for EBV lymphoproliferative disease”, Immunol Rev. 157:217-222 (1997); Dazzi, F. et al., “Donor lymphocyte infusions for relapse of chronic myeloid leukemia after allogeneic stem cell transplant: where we now stand” Exp Hematol. 27:1477-1486 (1999); Brodie, S. J. et al., “HIV-specific cytotoxic T lymphocytes traffic to lymph nodes and localize at sites of HIV replication and cell death”, J Clin Invest 105: 1407-1417 (2000)). Chimeric receptors linking antigen recognition domains, such as scFv or scTCR, to signaling domains derived from the TCR have proven straightforward to construct and highly effective at redirecting therapeutic T cells against desired targets (Eshhar, Z. “Tumor-specific T-bodies: towards clinical application” Cancer Immunol Immunother. 45:131-136 (1997); Wels, W. et al., “Biotechnological and gene therapeutic strategies in cancer treatment”, Gene. 159:73-80 (1995)). First generation receptors generally included the cytoplasmic tail of the TCR-ζ chain or the structurally and functionally similar FcεRI γ chain for signal transduction. However, these receptors proved limited by their inability to transduce supplementary costimulatory signals into T lymphocytes. As a result a newer generation of receptors that include the cytoplasmic tail of CD28 linked to ζ have been constructed.

Data from several groups has proven CD28-ζ receptors superior to otherwise identical ζ containing receptors (Abken, H. et al., “Chimeric T-cell receptors: highly specific tools to target cytotoxic T-lymphocytes to tumour cells”, Cancer Treat Rev. 23:97-112 (1997); Finney, H. M. et al., “Chimeric receptors providing both primary and costimulatory signaling in T cells from a single gene product”, J Immunol. 161:2791-2797 (1998); Hombach, A. et al., “Tumor-specific T cell activation by recombinant immunoreceptors: CD3 zeta signaling and CD28 costimulation are simultaneously required for efficient IL-2 secretion and can be integrated into one combined CD28/CD3 zeta signaling receptor molecule”, J Immunol. 167:6123-6131 (2001); Maher, J. et al., “Human T-lymphocyte cytotoxicity and proliferation directed by a single chimeric TCRzeta/CD28 receptor” Nat Biotechnol. 20:70-75 (2002)).

Although we observed enhanced function of a K^{b}-CD28-ζ receptor when compared with a K^{b}-ζ receptor in immortalized T cell hybridomas, we saw no functional difference when primary T lymphocytes were transduced with these receptors. We hypothesized that this resulted from the poor expression we observed with the K^{b}-CD28-ζ receptors. We now identify a dileucine motif in the cytoplasmic tail of murine CD28 that limits chimeric receptor expression and function. Two classes of dileucine motifs have been characterized, containing either [AspGlu]XaaXaaXaaLeu[LeuIle] (SEQ ID Nos.2-5) or AspXaaXaaLeuLeu (SEQ ID NO. 1) canonical sequences (Bonifacino, J. S. and Traub, L. M. “Signals for Sorting of Transmembrane Proteins to Endosomes and Lysosomes”. Annu Rev Biochem. 72:395-447 (2003)). The sequence we identified in the murine CD28 tail, SerArgArgAsnArgLeuLeu (SEQ ID No. 8), lacks the upstream negative charge typical of these motifs. AspXaaXaaLeuLeu (SEQ ID NO. 1) motifs bind to the GGA family of ARF-dependent clathrin adaptors and are intolerant of mutations of the upstream aspartic acid or of the twin leucines. In contrast the [AspGlu]XaaXaaXaaLeu[LeuIle] motif (SEQ ID Nos.2-5), which binds the AP-1, AP-2 and/or AP-3 family of adaptors, have more varied sequences at the amino end and may also contain either a leucine or isoleucine at the carboxyl end. The CD28 dileucine motif therefore falls into this latter class. Indeed the SerArgArgAsnArgLeuLeu CD28 motif (SEQ ID No. 8) resembles the positively charged ArgArgThrProSerLeuLeu (SEQ ID No. 10), ProArgGlySerArgLeuLeu (SEQ ID NO. 11), and SerGluArgArgAsnLeuLeu (SEQ ID No. 12) motifs of GLUT4, RAP, and VAMP4 respectively (Bonifacino, J. S. and Traub, L. M. “Signals for Sorting of Transmembrane Proteins to Endosomes and Lysosomes”. Annu Rev Biochem. 72:395-447 (2003)). The positive charge provided by the arginine residues in these dileucine motifs contrasts with the canonical negatively charged glutamic or aspartic acid residue, and is believed to influence the destination of these proteins with internalization (Sandoval, I. V. et al., “Distinct reading of different structural determinants modulates the dileucine-mediated transport steps of the lysosomal membrane protein LIMPII and the insulin-sensitive glucose transporter GLUT4”, J Biol Chem. 275:39874-39885 (2000)).

A positively charged dileucine motif, SerLysArgSerArgLeuLeu (SEQ ID No. 9), homologous to the murine sequence that we studied is present in the cytoplasmic tail of human CD28. We would therefore expect that mutations in the human motif would similarly improve the expression of human chimeric receptors that include the CD28 signaling chain.

Our results with chimeric receptors imply that the non-canonical dileucine motif in CD28 is biologically functional. The role of this dileucine motif in CD28 itself, however, is unclear. Although with our chimeric receptors we observe enhanced basal expression after disrupting the dileucine motif, in many other molecules dileucine mediated internalization is only apparent in restricted circumstances. For example, the SDKQTLL sequence of CD3γ requires serine phosphorylation to mediate internalization of the T-cell receptor complex (von Essen, M. et al., “The CD3 gamma leucine-based receptor-sorting motif is required for efficient ligand-mediated TCR down-regulation”, J Immunol. 168:45194523 (2002)). Potentially this motif is only accessible to membrane associated sorting proteins after a conformational shift induced by phosphorylation. It seems likely that the dileucine motif of native CD28 is likewise normally inaccessible, only mediating internalization in select circumstances, such as after activation when CD28 rapidly downmodulates. Placement of the CD28 dileucine motif outside of its native context, such as in chimeric receptors, may inadvertently expose it to the protein-sorting machinery and thereby constitutively activate its function.

The presence of a functional dileucine motif would be expected to limit signaling through chimeric receptors. Indeed, similar motifs present in the tails of CD3γ and CD4 are known to restrict TCR activity (Pitcher, C. et al., “Cluster of differentiation antigen 4 (CD4) endocytosis and adaptor complex binding require activation of the CD4 endocytosis signal by serine phosphorylation”, Mol Biol Cell. 10:677-691 (1999); Dietrich, J. et al., “Regulation and function of the CD3gamma DxxxLL motif: a binding site for adaptor protein-1 and adaptor protein-2 in vitro”, J Cell Biol. 138:271-281 (1997); Letourneur, F. and Klausner, R. D. “A novel di-leucine motif and a tyrosine-based motif independently mediate lysosomal targeting and endocytosis of CD3 chains”, Cell. 69:1143-1157 (1992)). Our results clearly demonstrate that inactivation of the CD28 dileucine motif increases expression of and strongly upregulates chimeric receptor mediated proliferation, cytokine production, and cytolysis. Although the location of the dileucine motif is important for its function, dileucine motifs may be located either membrane proximally or more C-terminal. Switching the CD28 and ζ modular domains of the CD28-ζ receptor may therefore not be adequate to inactivate it. Indeed we and others have shown even poorer expression of receptors that include a ζ-CD28 tail compared with the CD28-ζ tail present in the receptors studied here (Geiger, T. L. et al., “Integrated src kinase and costimulatory activity enhances signal transduction through single-chain chimeric receptors in T lymphocytes”, Blood 98: 2364-2371 (2001); Finney, H. M. et al., “Chimeric receptors providing both primary and costimulatory signaling in T cells from a single gene product”, J Immunol. 161:2791-2797 (1998)). In contrast to moving the CD28 domain, mutagenically inactivating it should more uniformly improve receptor potency.

In addition to demonstrating improved functional characteristics of dileucine-mutated receptors, we for the first time show that RMTC may be used to selectively target antigen-specific CD8⁺ T cells in-vivo. Approaches to selectively tolerize the alloantigen-specific or minor histocompatability antigen-specific T cells that mediate transplant rejection or graft-versus-host disease, such as the veto effect or the use of tolerizing regimens of antigen, are limited and have yet to be clinically validated (Wraith, D. et al., “Antigen recognition in autoimmune encephalomyelitis and the potential for peptide mediated immunotherapy”, Cell. 59:247-255 (1989); Fink, P. J. et al., “Veto Cells”, Ann Rev Immunol. 6:115 (1988); Reich-Zeliger, S. et al., “Anti-third party CD8+ CTLs as potent veto cells: coexpression of CD8 and FasL is a prerequisite” Immunity 13:507-515 (2000)). Retargeting RMTC against pathologic T cells represents a promising new therapeutic possibility. We observed improved in vivo cytolysis using K^{b}-CD28[L→G]-ζ compared with K^{b}-CD28-ζ RMTC, demonstrating that the mutated receptor is more potent and would likely be preferable for therapeutic use. Further studies, however, will be required to determine how these RMTC may be optimally applied to induce transplantation tolerance.

Various publications, patent applications and patents are cited herein, the disclosures of which are incorporated by reference in their entireties.

We claim:

1. A modified chimeric receptor comprising a chimeric receptor having a dileucine motif in its intracellular portion, wherein said modified chimeric receptor has a disruption in said dileucine motif.

2. The modified chimeric receptor of claim 1, wherein said dileucine motif has a sequence selected from the group consisting of SEQ ID No. 1 to SEQ ID No. 14.

3. The modified chimeric receptor of claim 1 wherein said dileucine motif is derived from a CD28 protein.

4. The modified chimeric receptor of claim 3 wherein said CD28 protein is a human CD28 protein and wherein said dileucine motif has the sequence set forth in SEQ ID No. 9.

5. The modified chimeric receptor of claim 3 wherein said CD28 protein is a murine CD28 protein and wherein said dileucine motif has the sequence set forth in SEQ ID No. 8.

6. The modified chimeric receptor of claim 1 wherein said modified chimeric receptor is a T-cell receptor.

7. The modified chimeric receptor of claim 1 wherein said disruption comprises an addition of at least one amino acid within said dileucine motif.

8. The modified chimeric receptor of claim 1 wherein said disruption comprises a deletion of at least one amino acid within said dileucine motif.

9. The modified chimeric receptor of claim 1 wherein said disruption comprises a substitution of at least one amino acid within said dileucine motif.

10. The modified chimeric receptor of claim 1 wherein said disruption comprises a substitution of at least one leucine within said dileucine motif.

11. A method for increasing the capacity of a chimeric receptor having a dileucine motif in its intracellular portion to accumulate on a cell comprising disrupting said dileucine motif.

12. A CD28 protein or portion thereof having a dileucine motif, wherein said CD28 protein has a disruption in said dileucine motif.

13. The CD28 protein or portion thereof of claim 12 wherein said CD28 protein is a murine protein.

14. The CD28 protein or portion thereof of claim 13 wherein said dileucine motif has the sequence set forth in SEQ ID No. 8.

15. The CD28 protein or portion thereof of claim 12 wherein said CD28 protein is a human protein.

16. The CD28 protein or portion thereof of claim 15 wherein said dileucine motif has the sequence set forth in SEQ ID No. 9.

17. A cell having on its membrane at least one modified chimeric receptor comprising a chimeric receptor having a dileucine motif in its intracellular portion, wherein said modified chimeric receptor has a disruption in said dileucine motif.

18. The cell according to claim 17, wherein said cell is a T-cell.

19. The cell according to claim 18, wherein said modified chimeric receptor is a T-cell receptor.
